(19) United States
(12) Patent Application Publication
(10) Pub. No.: US 2007/0034772 A1
(43) Pub. Date: Feb. 15, 2007

(54) Image sensor chip package

(75) Inventors: Steven Webster, Miao-li (TW); Ying-Cheng Wu, Miao-li (TW)

Correspondence Address: PCE INDUSTRY, INC.;ATT. CHENG-JU CHIANG JEFFREY T. KNAPP, 458 E. LAMBERT ROAD, FULLERTON, CA, 92835, US

(73) Assignee: ALTUS TECHNOLOGY INC., Miao-li Hsien (TW)

(21) Appl. No.: 11/448,314

(22) Filed: Jun. 7, 2006

(30) Foreign Application Priority Data
Aug. 12, 2005 (CN) 200510036587.2

(51) Int. Cl. C12Q 1/68

(52) U.S. Cl. 250/200

(57) ABSTRACT

A digital camera module includes a barrel (10), a seat (20) and an image sensor chip package (30) in accordance with a preferred embodiment is shown. The image sensor chip package includes a carrier (32), a chip (34), a number of bonding wires (36) and a cover (38). The carrier includes a base (24). The chip is mounted on the base and has an active area. The second conductive means electronically connects the chip and the conductive means. An adhesive means is applied around the active area of the chip. The transparent cover is mounted to the base of the carrier. The cover adheres to the carrier with the adhesive means and defines a sealing space (37) for sealing the active area of the chip therein. The active area of the chip is sufficiently protected from pollution by the small volume of the sealing space.

### CROSS-REFERENCE TO RELATED APPLICATIONS

This application is related to a co-pending U.S. patent applications (Attorney Docket No.US7595), entitled “IMAGE SENSOR CHIP PACKAGE”, by Steven Webster et al. Such application has the same assignee as the present application and has been concurrently filed herewith. The disclosure of the above identified application is incorporated herein by reference.

### TECHNICAL FIELD

The present invention generally relates to an IC (integrated circuit) chip package and, more particularly, to a digital camera module with an image sensor chip package.

### BACKGROUND

Image sensors are widely used in digital camera modules in order to convert the optical image data of an object into electrical signals. In order to protect the image sensor from contamination or pollution (i.e. from dust or water vapor), the image sensor is generally sealed in a structural package.

A typical image sensor chip package (not labeled) is illustrated in FIG. 6. The image sensor chip package is constructed to include a plurality of conductors 130, a base 146, a chip 152 and a cover 158. The base 146 includes a bottom portion 148 and four sidewalls 149. The bottom portion 148 and the sidewalls 149 cooperatively form a space 150. Each conductor 130 includes a first conductive portion 140, a second conductive portion 142 and a third conductive portion 144. The first and second conductive portions 140, 142 are respectively mounted on two sides of the bottom portion 148. The third conductive portion 144 runs through the bottom portion 148 so as to connect the first and second conductive portions 140, 142. The chip 152 includes a number of pads 154 formed thereon. The chip 152 is received in the space 150 and fixed on the base 146 by an adhesive glue 160. A number of bonding wires 156 are provided to connect the pads 154 and the first conductive portion 140 of the conductors 130. The cover 158 is transparent and secured to the top of the sidewalls 149 via an adhesive glue 162, thereby hermetically sealing the space 150 and allowing light beams to pass therethrough.

In the process of forming the conductors 130, a number of interconnection holes 166 are defined in the base 146. The first portions 140 and the second portions 142 are formed by means of plating metal. The third portions 136 are formed by filling molten metal into the interconnection holes 166 and then allowing it to solidify. Accordingly, the first portion 140, the second portion 142 and the third portion 136 are electronically connected with each other. It is obvious that this method of forming the conductors 130 is complex and as a result it is also expensive. Furthermore, after the conductors 130 are formed, water vapor can enter the space 150 via the interconnection holes 166. Thus, the chip 152 may be polluted and the conductors 130 may be damaged.

In addition, the relative large volume of the image sensor chip package results in more dust-particles adhering to the cover 158, the bottom board 1462 and the sidewalls 1464 of the base 146. Thus, more dust-particles will drop onto the chip 152. The dust-particles obscure the optical path and produce errors in the image sensing process. Accordingly, the quality and/or reliability of the image sensor chip package 100 can be effected. Moreover, the bonding wires 156 exposed in the space 150 lack protection and may thus easily be damaged by dust-particles entering the space 150.

Therefore, a new image sensor chip package is desired in order to overcome the above-described shortcomings.

### SUMMARY OF THE INVENTION

One embodiment of a chip package includes a carrier, an image sensor chip, a number of wires, an adhesive means and a transparent cover. The carrier includes a base and a leadframe. The base has a cavity therein. The leadframe includes a plurality of conductive pieces embedded in the base and are spaced from each other. The image sensor chip is mounted on the base and received in the cavity. The image sensor has a photosensitive area. The wires each electronically connect the image sensor chip and one corresponding conductive piece of the carrier. The adhesive means surrounds the image sensor chip and at least partially covers all the wires. The transparent cover is mounted to the carrier, the cover is adhered to the carrier with the adhesive means in a manner so as to define a sealing space for a photosensitive area of the image sensor chip therein, and the base and the cover allows one end of the conductive pieces to be exposed out therefrom.

Other advantages and novel features will become more apparent from the following detailed description when taken in conjunction with the accompanying drawings.

### BRIEF DESCRIPTION OF THE DRAWINGS

Many aspects of the present image sensor chip package can be better understood with reference to the following drawings. The components in the drawings are not necessarily drawn to scale, the emphasis instead being placed upon clearly illustrating the principles of the image sensor chip package. Moreover, in the drawings, like reference numerals designate corresponding parts throughout the several views.

FIG. 1 is a schematic, cross-sectional view of a digital camera module with an image sensor chip package according to a preferred embodiment;

FIG. 2 is a schematic, top plan view of the leadframe of FIG. 1;

FIG. 3 is a cross-sectional view of the leadframe in FIG. 2 along a line III-Ill;

FIG. 4 is a schematic, top view of the carrier shown in FIG. 1;

FIG. 5 is a schematic, bottom view of the carrier shown in FIG. 4; and

FIG. 6 is a schematic, cross-sectional view of a typical image sensor chip package.

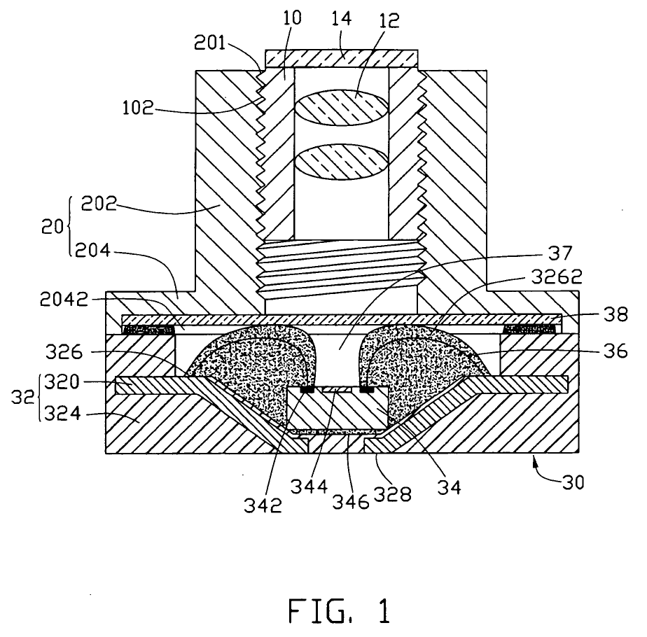

### DETAILED DESCRIPTION OF THE PERFERRED EMBODIMENTS

Referring to FIG. 2, a digital camera module includes a barrel 10, a seat 20 and an image sensor chip package 30 in accordance with a preferred embodiment. The image sensor chip package 30 includes a carrier 32, a chip 34, a number of bonding wires 36 and a cover 38.

The barrel 10 is substantially a hollow cylinder with two open ends so that light can be transmitted therethrough. Several lens elements 12 are disposed in the barrel 10, and receive incoming light that enters from the outside. The barrel 10 has an outer thread 102 defined in an outer periphery wall thereof. A glass board 14 is disposed before the lens elements 12 and covers one end of the barrel 10. As such, the glass board 14 protects the lens elements 12 from being scraped or otherwise abraded and keeps dust from entering the system.

The seat 20 includes a seat body 202 and a flange 204 formed together. The seat body 202 is a hollow cylinder. The flange 204 is formed at a bottom end of the seat body 202. The flange 204 has a rectangular cavity 2042 defined in a middle thereof opposite to the seat body 202. The rectangular cavity 2042 communicates with the seat body 202 so that light can be transmitted therethrough. An outer diameter of the seat body 202 is smaller than an edge of the flange 204 so that a step is formed at a connection between them. An inner periphery wall of the seat body 202 defines an inner thread 201, for engaging with the outer thread 102 of the barrel 10.

The carrier 32 of the image sensor chip package 30 includes a leadframe 320 and a plastic base 324. The leadframe 320 and the plastic base 324 cooperatively form the carrier 32 by insert-molding.

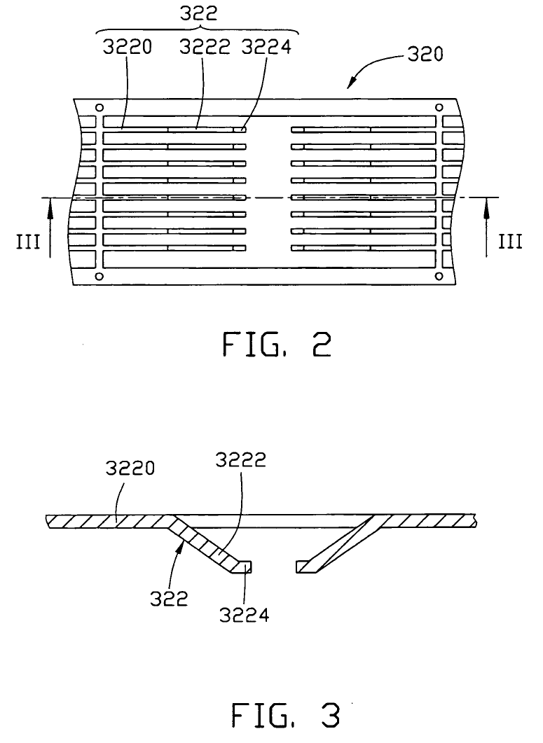

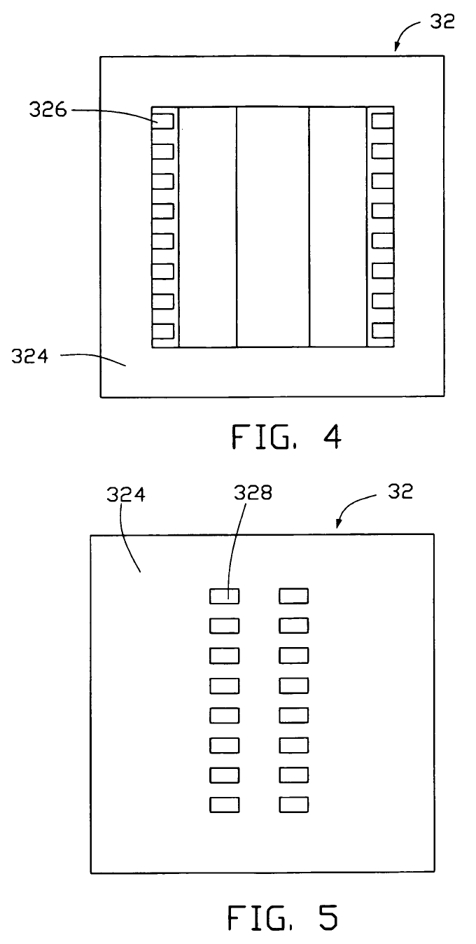

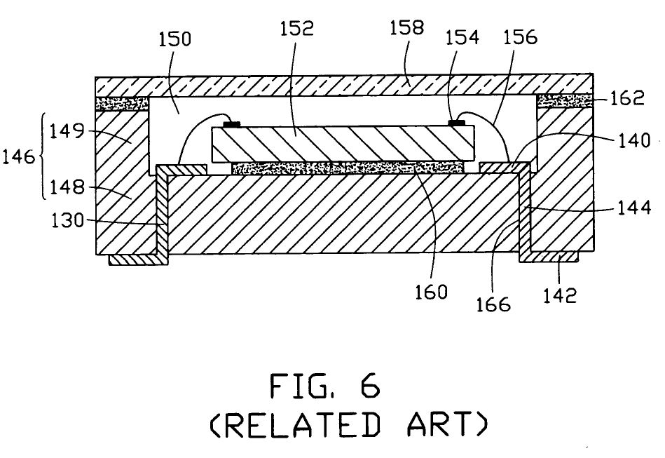

Referring to FIGS. 3 and 4, the leadframe 320 includes a number of conductive pieces 322 made of metal. The leadframe 320 forms the conductive pieces 322 by either punching or etching on a metal sheet. Each of the conductive pieces 322 includes a first terminal portion 3220, a second connecting portion 3222 and a third terminal portion 3224, the second connecting portion 3222 interconnecting the first and third terminal portions 3220, 3224. The first and third portions 3220, 3224 are spaced apart and aligned in parallel to each other. The second portion 3222 is slanted relative to the first and third portions 3220, 3224. Correspondingly, the conductive pieces 322 are divided into two groups. The two groups are symmetrically arranged and the conductive pieces in the same group are parallel to and spaced from each other.

During insert-molding, the plastic base 324 partially encloses the upper and lower surfaces of the leadframe 320. One end of each of the first portions 3220 is exposed, thus forming a plurality of upper pads 326. A distal end of each of the third portions 3224 is exposed, thus forming a number of lower pads 328. After insert-molding, the leadframe 320 is cut along two sides. The carrier 32 is formed with a trapezoidal cavity. Referring to FIGS. 4 and 5 the upper pads 326 are electronically connected to the image sensor chip 34. The lower pads 328 are connected to a printing circuit board (PCB), for electronically connecting the leadframe 320 to the PCB so as to transmit electric signals out of the image sensor chip package 30.

The image sensor chip 34 is received in the trapezoidal cavity, and is adhered to the bottom of the carrier 32 via an adhesive glue 346. A top surface of the image sensor chip 34 is arranged with a photosensitive area 344 and a number of chip pads 342 around the photosensitive area 344. Alternatively, the adhesive glue 346 can be replaced by any other appropriate adhesive means.

The bonding wires 36 can be made of a conductive material such as gold or aluminum alloy. One end of each wire 36 is connected/joined with a respective chip pad 342 of the image sensor chip 34, and the other end of the wire 36 is connected/joined with a respective upper pad 326 forming by the first portions 3220 of the leadframe 320.

The cover 38 is transparent and is laid over the image sensor chip 34 which receives light transmitted through the cover 38. The cover 38 is adhered on the base 324 by glue (not labeled) and therefore seals the image sensor chip 34 in the cavity of the base 324.

The adhesive glue 3262, such as a silicone, epoxy, acrylic, or polyamide adhesive, is applied around the photosensitive area 344 of the image sensor chip 34. The adhesive glue 3262 surrounds all of the bonding wires 36 and covers the first portions 3220, and the inner surface of the carrier 32. The adhesive glue 3262 also fixes the cover 38 and defines a small sealing space 37 configured to seal the photosensitive area 344 therein. The bonding wires 36 and the adhesive glue 3262 are received in the carrier 32. It can be seen that the photosensitive area 344 of the image sensor chip 34 is sufficiently protected from outside pollution due to the small volume of the sealing space 37. The bonding wires 36 are protected by the adhesive glue 3262, and the leadframe 320 is protected by the base 324.

In assembly, several lens elements 12 are received in the barrel 10. The outer thread 102 of the barrel 10 engages with the inner thread 201 of the seat 20, whereby the barrel 10 and the seat 20 are connected with each other. The seat 20 is then mounted on the image sensor chip package 30 by welding/glue, with the image sensor 34 aligning with the lens elements 12. At the same time, the cover 38 is received in the rectangular cavity 2042. The assembly process of the digital camera module is thus completed.

In an alternative embodiment, the arrangement of the conductive pieces of the leadframe 320 can be changed so long as the conductive pieces 322 are spaced from each other. The second portion 3222 can be perpendicular to the first and third portions 3220, 3224 rather than being slanted. The base 324 can be of another shape such as cylinder-shaped, or column-shaped with a pentagonal or hexagonal cross-section.

In the above embodiments, the adhesive glue 3262 may only be disposed around the photosensitive area 344 so as to define a sealing space for protecting the photosensitive area 344. One main advantage of the digital cameral module with this image sensor chip package is its reliability and high image quality.

It is believed that the present embodiments and their advantages will be understood from the foregoing description, and it will be apparent that various changes may be made thereto without departing from the spirit and scope of the invention or sacrificing all of its material advantages, the examples hereinbefore described merely being preferred or exemplary embodiments of the invention.

We claim:

1. An image sensor chip package comprising:
a carrier comprising a base and a leadframe, the base having a cavity therein, the leadframe comprising a plurality of conductive pieces, the conductive pieces of the leadframe being embedded in the base and spaced from each other;
an image sensor chip mounted on the base and received in the cavity, the image sensor having a photosensitive area;
a plurality of wires each electronically connecting the image sensor chip and a corresponding one of the conductive pieces of the carrier;
an adhesive means applied around the image sensor chip and at least partially covering all the wires; and
a transparent cover mounted to the carrier, the cover adhered to the carrier with the adhesive means and configured in a manner so as to define a sealing space for a photosensitive area of the image sensor chip therein, and the base and the cover allowing one end of the conductive pieces to be exposed out therefrom.

2. The image sensor chip package as claimed in claim 1, wherein the leadframe is manufactured by punching or etching on a metal sheet, the base being made of plastic materials.

3. The image sensor chip package as claimed in claim 1, wherein the each of the conductive pieces of the leadframe comprises a first portion, a second portion and a third portion, the first and second portions are parallel to and spaced from each other, the second portion is slanted relative to the first and third portions.

4. The image sensor chip package as claimed in claim 1, wherein the each of the conductive pieces of the leadframe comprises a first portion, a second portion and a third portion, the first and second portions are parallel to and spaced from each other, the second portion is slanted relative to the first and third portions.

5. The image sensor chip package as claimed in claim 1, wherein the carrier is formed so as to allow it to enclose the leadframe in the base, and the cavity is trapezoidal in shape.

6. The image sensor chip package as claimed in claim 1, wherein the cover is adhered to the carrier by adhesive.

7. A digital camera module comprising:
a barrel,
an image sensor chip package, the barrel mounted on the image sensor chip package, the image sensor chip package comprising:
a carrier comprising a conductive means and a base, the conductive means forming a plurality of pads outside the base;
an image sensor chip mounted on the base, the image sensor chip having a photosensitive area and a plurality of chip pads around the photosensitive area;
a plurality of wires each electronically connecting a corresponding one of the chip pads of the image sensor chip and the conductive means;
an adhesive means applied around the photosensitive area of the image sensor chip; and
a transparent cover mounted to the base of the carrier, the cover adhered with the adhesive means, thereby defining a sealing space for sealing the photosensitive area of the image sensor chip therein.

8. The digital camera module as claimed in claim 7, wherein the adhesive means surrounds the wires.

9. The digital camera module as claimed in claim 7, wherein the base of the carrier has a cavity therein, the conductive means comprises a plurality of conductive pieces embedded in the base and spaced from each other.

10. The digital camera module as claimed in claim 9, wherein the leadframe is manufactured by punching or etching on a metal sheet, the base is made of plastic materials.

11. The digital camera module as claimed in claim 9, wherein the carrier is manufactured by insert-molding.

12. The digital camera module as claimed in claim 9, wherein each of the conductive pieces of the conductive means comprises a first portion, a second portion and a third portion, the first and third portions are parallel to and spaced from each other, and the second portion is slanted relative to the first and third portions.

13. The digital camera module as claimed in claim 7, further comprising a seat, the seat includes a seat body and a flange, the seat body is connect with the barrel by thread, and the flange is mounted on the image sensor chip package.

14. The digital camera module as claimed in claim 7, wherein the barrel includes a glass board, and the glass board is disposed on one end of the barrel.

15. A digital camera module comprising:
a barrel,
an image sensor chip package, the barrel mounted on the image sensor chip package, the image sensor chip package comprising:
a carrier,
an image sensor chip mounted on the carrier, the image sensor chip having a plurality of chip pads;
a plurality of wires each electronically connecting a corresponding one of the chip pads of the image sensor chip and the conductive means;
an adhesive means applied around the photosensitive area of the image sensor chip; and
a transparent cover mounted to the base of the carrier, the cover adhered with the adhesive means and configured in a manner so as to allow it to define a sealing space for sealing the photosensitive area of the image sensor chip therein.

16. The digital camera module as claimed in claim 15, wherein the adhesive means surrounds the wires.

17. The digital camera module as claimed in claim 15, wherein the base of the carrier has a cavity therein, the conductive means comprises a plurality of conductive pieces embedded in the base and spaced from each other.

18. The digital camera module as claimed in claim 17, wherein each of the conductive pieces of the conductive means comprises a first portion, a second portion and a third portion, the first and third portions are parallel to and spaced from each other, the second portion is slanted relative to the first and third portions.

19. The digital camera module as claimed in claim 15, further comprising a seat, the seat includes a seat body and a flange, the seat body is connect with the barrel by thread, and the flange is mounted on the image sensor chip package.
